(19) United States
(12) Patent Application Publication
(10) Pub. No.: US 2005/0277968 A1
(43) Pub. Date: Dec. 15, 2005

(54) Percutaneous surgical device

(76) Inventor: Doohi Lee, Plano, TX (US)

Correspondence Address: Doohi Lee, 5508 Ash Creek Lane, Plano, TX, 75093, US

(21) Appl. No.: 10/856,672

(22) Filed: May 28, 2004

(51) Int. Cl. A61B 17/32

(52) U.S. Cl. 606/170

(57) ABSTRACT

A percutaneous surgical device comprises a tip with a side opening, a protective sheath coupled to the tip, with the sheath and tip house at least one surgical instrument, and a handle coupled to the sheath with at least one control means for controlling a moving element for further controlling the surgical instrument to be exposed through the side opening of the tip.

### BACKGROUND

The present disclosure relates generally to surgical devices, and more particularly to a percutaneous surgical device having an opening for operating a retractable surgical instrument.

In various percutaneous surgical procedures, surgery is performed with a surgical instrument below the skin in a predetermined part of the body. The surgical instrument is typically inserted below the skin through a small incision at the skin surface.

Typically, if the surgical location is close to the skin surface, the surgical instrument is simply inserted directly through the small incision at the skin surface without any protection mechanism. However, if the surgical location is not close to the skin surface, i.e. if the surgical instrument must travel for a non-trivial distance between the small incision at which the surgical instrument is originally inserted and a specified percutaneous surgical location, the surgical instrument must, in many cases, be protected throughout the path of travel. One such case is when the surgical instrument has sharp edges that may damage other body tissues and structures during the travel. Another such case is when the surgical instrument must not be contaminated before a surgical procedure is performed. In both cases, the corresponding surgical procedures necessitate, that extreme care must be taken when the surgical instrument is introduced into, travel through, or withdrawn from the body. An exposed surgical instrument may cause not only undesirable damage to body tissues and structures, but also contamination prior to the relevant surgical procedures.

Such issues have previously been addressed by designs that introduce, for the surgical instrument, a protective sleeve through which, for example, a blade slides into and out of position for protection. However, such designs are not very practical in surgical procedures, as the blade slides into and out of exposure at the same dimension as, and not at an angle to, the protective sleeve. As such, the blade's reach and usefulness may be severely limited. This issue is particularly acute, when the designs must travel through a long, narrow tunnel. In various percutaneous surgeries, the surgical instrument must travel through a long, narrow hollow tunnel and perform surgical procedure at the inner side wall thereof. It may be very difficult, as an example, to use the sharp edge of the surgical instrument effectively, if the blade were to cut a particular inner side wall of, say, a long, narrow tube, simply because the long, narrow tube may not allow the operator of the blade to introduce the cutting edge at a non-trivial angle incident to the inner side wall because of maneuver limitations.

In order to introduce instrument at an angle different from the angle of travel, especially in a long, narrow hollow tunnel, various complex engineering solutions such as a combination of pulleys, pivots and railings may need to be introduced. However, since such engineering solutions may be either very space-consuming in order to be mechanically effective or costly to be space-efficient, it is both impractical and costly to implement current engineering solutions in surgical devices.

Desirable in the art of surgical devices are improved designs that allow a surgical instrument to be introduced at an angle different from the angle of travel with simple engineering solutions, thereby increasing surgical coverage and enhancing practicality. In addition, additional designs that allow the surgical instrument to be flexible, thereby allowing the surgical instrument to travel through a non-linear hollow tunnel, are also desirable.

### SUMMARY

In view of the foregoing, this disclosure provides a method and system to allow a surgical instrument to be introduced at an angle different from the angle of travel with simple engineering solutions, and to be flexible enough such that it may travel through a non-linear path.

In one example, a percutaneous surgical device is provided with a side opening that allows a surgical instrument to be introduced at an angle different from the angle of travel. The percutaneous surgical device comprises a tip with a side opening, a protective sheath coupled to the tip, with the sheath and tip housing at least one surgical instrument, and a handle coupled to the sheath with at least one control means for controlling a moving element for further controlling the surgical instrument to be exposed through the side opening of the tip.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1A illustrates a percutaneous surgical device in accordance with one example of the present disclosure.

FIGS. 1B-1E present the side and top views of two side opening designs in accordance with examples of the present disclosure.

FIG. 2 presents five surgical instruments retractable through the side opening in accordance with examples of the present disclosure.

FIG. 3 presents a cross section of the handle in accordance with one example of the present disclosure.

FIGS. 4A-8B illustrate five examples of the surgical device in accordance with examples of the present disclosure.

FIGS. 9A-9D illustrate a surgical device having a flexible sheath in accordance with one example of the present disclosure.

### DESCRIPTION

This disclosure provides a detailed description of a percutaneous surgical device with a side opening that allows a surgical instrument to be introduced at an angle different from the angle of travel.

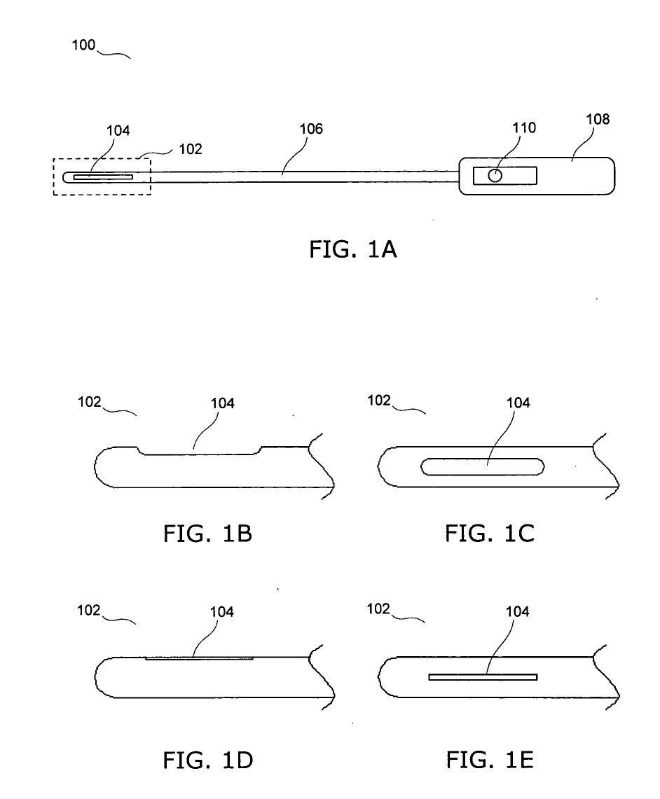

FIG. 1A illustrates a percutaneous surgical device 100 in accordance with one example of the present disclosure. The surgical device 100 includes a tip 102 with a side opening 104, a protective sheath 106, a handle 108 with a control mechanism such as an adjustable knob 110. The tip 102 may be a disposable module that may be attached to the protective sheath 106. The protective sheath 106 houses moving surgical instruments, to be further discussed later. It is understood that the tip and the sheath can be an integrated piece or two separate components that are coupled together seamlessly. While the following illustration uses some specific surgical device designs as examples, it is understood that some components that are depicted within the tip can be easily altered to be included in the sheath instead of the tip.

FIGS. 1B-1C present the side and top views of a first design of the tip 102 with a side opening in accordance with one example of the present disclosure. The side opening is a slot, through which at least one surgical instrument may be exposed and from which at least that surgical instrument may be retracted. FIGS. 5C-1D present the side and top views of another design of the tip 102 in accordance with another example of the present disclosure. The side opening is a slit, through which at least one smaller surgical instrument may be exposed and from which at least that smaller surgical instrument may be retracted. The variation in the size of the side opening accommodates various surgical instruments as long as the side opening is just large enough for the surgical instrument to travel therethrough. In one example, the first side opening may accommodate an extracting instrument that may extract body samples through the side opening. In another example, the second side opening may accommodate a cutting instrument (e.g. a blade) that may cut the side walls of a tunnel cavity through which the surgical device travels. While two side opening designs are presented in this disclosure, it is understood that designs other than a slot and a slit are also contemplated as long as their application and usage do not deviate from the spirit of this disclosure.

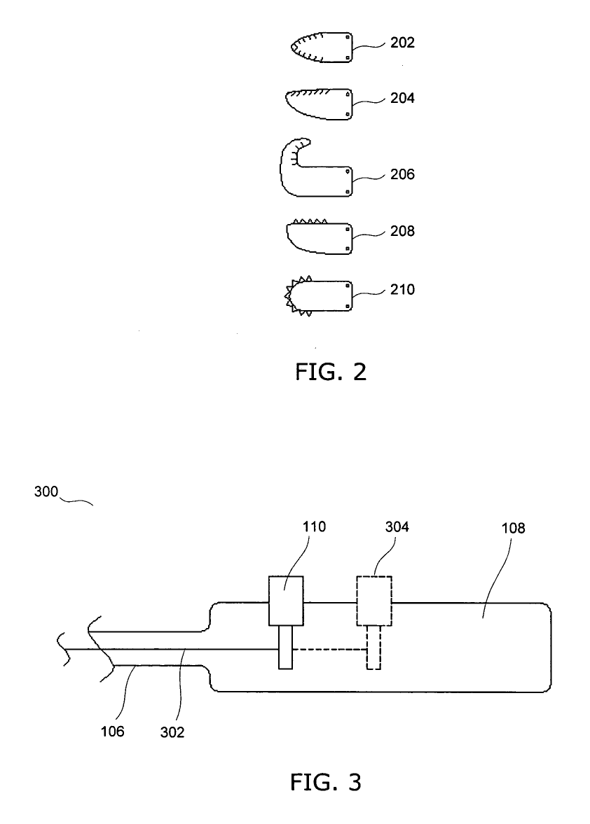

FIG. 2 presents several examples of surgical instruments retractable through the side opening in accordance with examples of the present disclosure. The assortment of surgical instruments includes a two-sided blade 202, a single-sided blade 204, a hook 206, a serrated blade 208, and a saw 210. For example, the blades 202 and 204 may be used for cutting. The hook 206 may be used for extracting and retrieving tissues, while the saw 210 may be used for taking apart hardened calcium deposits. It is however understood that the five surgical instruments presented in FIG. 2 are merely contemplated examples of how a surgical instrument may look like, and that other surgical instrument designs are contemplated without deviating from the spirit of this disclosure.

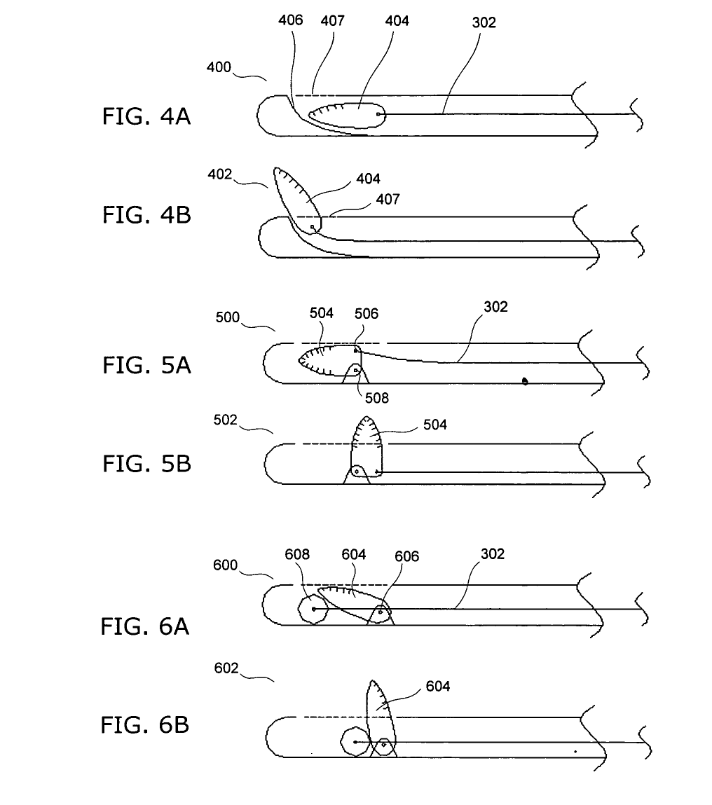

FIG. 3 presents a cross section 300 of the handle 108 in accordance with one example of the present disclosure. The handle 108 houses the adjustable knob 110, which provides the mechanism for controlling a surgical instrument attached thereto. In this example, the adjustable knob 110 pulls backward and pushes forward one end of a moving element 302, which is attached to the surgical instrument. The pull-push action of the adjustable knob 110 affects, through the moving element 302 that is housed inside the protective sheath 106, the movement of the surgical instrument. However, it will be illustrated that the other end of the moving element 302 need not be attached directly to the surgical instrument, but to other moving pieces that in turn affect the movement of the surgical instrument. The adjustable knob 110 can be locked into a particular position once decided by an operator so that the position of the surgical instrument whether exposed or unexposed will not be altered. Furthermore, a distance marking mechanism can be included so that the movement of the moving element can be measured, which in turn indicates the position information of the surgical instrument such as the extent to which the surgical instrument is exposed through the side opening.

With reference to FIG. 3, the adjustable knob 110 is parked at a given position before a surgical procedure. When the tip of the surgical device is in a predetermined place, the adjustable knob 110 slides to a new position 304 (as illustrated in dotted lines in FIG. 3), thereby pulling the moving element 302 towards the handle 108 and away from the tip (not shown) of the surgical device. By pulling the moving element 302 away from the tip of the surgical device, either the surgical instrument or other moving pieces housed inside the tip of the surgical device may be moved, thereby affecting the position and/or orientation of the surgical instrument. The movement may cause the instrument to be exposed.

As an example, the moving element 302 may be a wire (e.g. a music wire made from high carbon steel alloy). The wire may be advantageous in this design because of its dual characteristics of flexibility and rigidity, thereby allowing both push-pull actions in more than one dimension.

FIGS. 4A-8B illustrate examples showing the tip portion of the surgical device with an surgical instrument operated therewith. FIGS. 4A and 4B respectively present a safe position 400 and an exposed position 402 of a surgical instrument 404 in accordance one example of the present disclosure. While the surgical instrument 404 is illustrated in FIGS. 4A and 4B to be a blade, it is however understood by those skilled in the art that other surgical instruments may be used, including but not limited to those previously discussed, without deviating from the spirit of this disclosure.

The surgical instrument 404 is attached to the moving element 302. The attachment may be permanent or temporary, depending on various factors, including whether or not the solution is a disposable design. With the surgical instrument 404 in the safe position 400, the device is introduced into a patient's body through a small incision at the skin surface. The device may travel through the body safely, because the surgical instrument 404 is housed inside the tip of the device.

When the tip of the device reaches a particular surgical location, the adjustable knob is moved such that the moving element 302 begins to move. When the moving element 302 moves towards the tip of the device, it pushes the surgical instrument 404 onto and up a ramp 406. As the surgical instrument 404 is pushed farther up the ramp 406, it becomes more exposed through a side opening 407, thereby allowing it to be in direct contact with any surgical location. The surgical instrument 404 is now transitioned from the safe position 400 to the exposed position 402. The degree of exposure of the surgical instrument 404 may be determined by how far the moving element 302 is pushed towards the tip of the device. In turn, that distance is determined by how far the adjustable knob has traveled, according to FIG. 3. That distance may ultimately be controlled by gauges, as well as markings on the handle of the device indicating how much the moving element has or can be moved, thereby further indicating how much that movement has changed the surgical instrument's degree of exposure.

By contrast, when the moving element 302 moves away from the tip of the device, it pulls the surgical instrument 404 out of the side opening 407 and down the ramp 406. As the surgical instrument 404 is pulled down the ramp 406, it becomes less exposed through the side opening 407. When the surgical instrument 404 is no longer exposed through the side opening 407, it is considered to be in the safe position 400. At this time, the device may be withdrawn from the surgical location and out of the patient's body.

FIGS. 5A and 5B respectively present a safe position 500 and an exposed position 502 of a surgical instrument 504 in accordance with another example of the present disclosure. While the surgical instrument 504 is illustrated in FIGS. 5A and 5B to be a blade, it is however understood by those skilled in the art that other surgical instruments may be used, including but not limited to those previously discussed, without deviating from the spirit of this disclosure.

The surgical instrument 504 is attached to the moving element 302 by a securing mechanism such as a pin 506, while the surgical instrument 504 is also hinged to the inner wall of the tip portion through a securing mechanism such as a hinge 508. By using the hinge 508, the surgical instrument 504 may be pulled into or out of exposure. For example, if the moving element 302 moves away from the tip of the device, the surgical instrument 504 will move from the safe position 500 to the exposed position 502. By contrast, if the moving element 302 moves towards the tip of the device, the surgical instrument 504 will move from the exposed position 502 to the safe position 500. By pushing and pulling the adjustable knob at the rear end of the device, thereby pushing and pulling the moving element 302, the surgical instrument may be retractable and introduced at an angle different from the angle of travel.

FIGS. 6A and 6B respectively present a safe position 600 and an exposed position 602 of a surgical instrument 604 in accordance with another example of the present disclosure. The surgical instrument 604 is hinged to the inner wall of the tip through a securing mechanism such as a hinge 606. An activation component such as a wedge 608 resides underneath the surgical instrument 604, and is attached to the moving element 302. By sliding the wedge 608 underneath the surgical instrument 604, the surgical instrument 604 may swing into and out of exposure. For example, if the moving element 302 moves away from the tip of the device, the wedge 608 moves closer to the hinge 606, thereby causing the surgical instrument 604 to move from the safe position 600 to the exposed position 602. By contrast, if the moving element 302 moves towards the tip of the device, the wedge 608 moves away from the hinge 606, thereby causing the surgical instrument 604 to move from the exposed position 602 to the safe position 600. By pushing and pulling the adjustable knob at the rear end of the device, thereby pushing and pulling the moving element 302, the surgical instrument may be retractable and introduced at an angle different from the angle of travel.

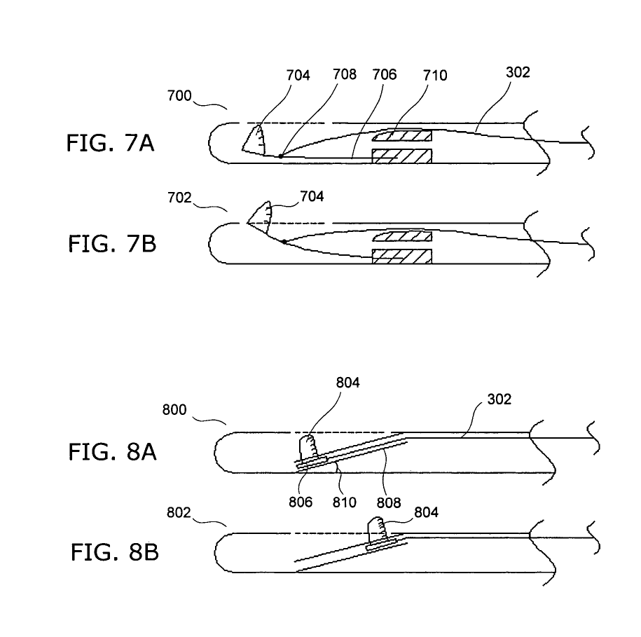

FIGS. 7A and 7B respectively present a safe position 700 and an exposed position 702 of a surgical instrument 704 in accordance with another example of the present disclosure. The surgical instrument 704 is attached to a bendable pole 706, which is further attached to the inner wall of the tip of the device. The bendable pole 706 is also connected, through a connector 708, to the moving element 302, which is elevated at a higher position by a pivot 710. By pulling and releasing the moving element 302, the bendable pole 706 may be bent, causing the surgical instrument 704 to swing into and out of exposure. For example, if the moving element 302 moves away from the tip of the device, the bendable pole 706 bends upwards, thereby causing the surgical instrument 704 to move from the safe position 700 to the exposed position 702. By contrast, if the moving element 302 moves towards the tip of the device, or is released from its tension if it is a string, the bendable pole 706 becomes horizontal, thereby causing the surgical instrument 704 to move from the exposed position 702 to the safe position 700. By pushing and pulling the adjustable knob, thereby pushing and pulling the moving element 302, the surgical instrument may be retractable and introduced at an angle different from the angle of travel. The pivot 710 is introduced to ensure that the moving element 302 is elevated such that the pulling of the moving element 302 will introduce a vertical force component at the connector 708, thereby causing the bendable pole 706 to bend.

FIGS. 8A and 8B respectively present a safe position 800 and an exposed position 802 of a surgical instrument 804 in accordance with another example of the present disclosure. In this example, the surgical instrument 804 is provided with an angled moving path. In this specific example, the surgical instrument 804 rests on a rail cart 806, which travels along a rail 808. The rail 808 is tilted at a non-trivial angle 810 such that as the rail cart 806 travels to the elevated end of the rail 808, the surgical instrument 804 becomes exposed. The rail cart 806 is attached to the moving element 302. For example, if the moving element 302 moves away from the tip of the device, the rail cart 806 travels to the elevated end of the rail 808, thereby causing the surgical instrument 804 to move from the safe position 800 to the exposed position 802. By contrast, if the moving element 302 moves towards the tip of the device, the rail cart 806 travels to the non-elevated end of the rail 808, thereby causing the surgical instrument 804 to move from the exposed position 802 to the safe position 800. By pushing and pulling the adjustable knob, thereby pushing and pulling the moving element 302, the surgical instrument may be retractable and introduced at an angle different from the angle of travel.

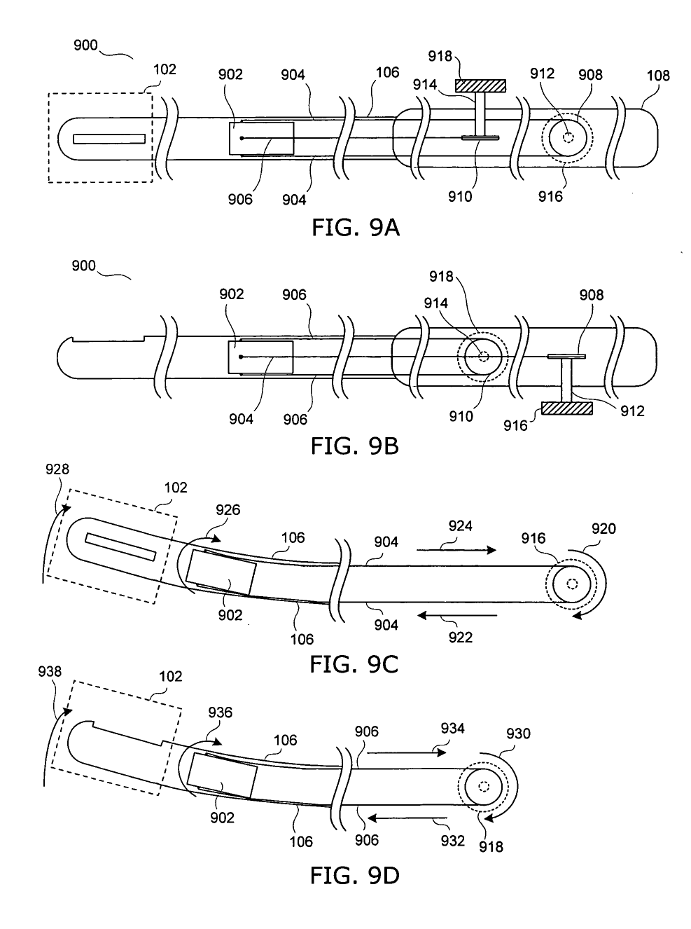

FIGS. 9A-9D illustrate a surgical device with a flexible sheath for changing an orientation of the tip in accordance with another example of the present disclosure. FIG. 9A illustrates a top view of a flexible percutaneous surgical device 900. The flexible percutaneous surgical device 900 is modified from the percutaneous surgical device 100 such that the tip of the device may be curved relative to the handle of the device. Various design elements from the percutaneous surgical device 100 are shown, including the tip 102, the protective sheath 106 and the handle 108. For the ease of illustration, the adjustable knob 110 is not shown, although it is understood that it may be required for the proper operation of the flexible percutaneous surgical device 900. It is understood that the protective sheath 106 is flexible, and may be made of elastic materials such as low density polyethylene. The flexible percutaneous surgical device 900 further includes a curvature guide 902, to which the deflection wires 904 and 906 are attached. The deflection wire 904 wraps around a wheel 908, which is housed inside the handle 108. The deflection wire 906 wraps around a wheel 910, which is also housed inside the handle 108. The wheel 908 and 910 may be turned by rotating the axes 912 and 914, respectively. The axes 912 and 914 may be turned, from outside of the handle 108, by the turning knobs 916 and 918. It is noteworthy that in this top view, the turning knob 916 is behind the handle 108, and as such is represented in dotted lines. It is further noteworthy that in this top view, the deflection wire 906 attached to one side of the curvature guide 902 wraps around the wheel 910 and attaches to the other side of the curvature guide 902 (not shown).

FIG. 9B illustrates a side view of the flexible percutaneous surgical device 900 in accordance with one example of the present disclosure. The side view may be obtained if the flexible percutaneous surgical device 900 as represented in the top view in FIG. 9A is viewed, from the bottom of the page, towards the top of the page. This side view shows that the deflection wire 906 attaches to one side of the curvature guide 902, wraps around the wheel 910, and attaches to the other side of the curvature guide 902. Because the view angle in FIG. 9B is different from the view angle in FIG. 9A, the axis 914 and the turning knob 918 are now behind the handle 108, and therefore are shown in dotted lines. It is noteworthy that in this side view, the axis 912 and the turning knob 916 may be seen. It is further noteworthy that in this side view, the deflection wire 904 attached to one side of the curvature guide 902 wraps around the wheel 908 and attaches to the other side of the curvature guide 902 (not shown). It will be evident, through the following explanations, how the flexible percutaneous surgical device 900 may be curved. This curvature may enable the flexible percutaneous surgical device 900 to travel through a non-linear path, and therefore may allow the device to reach areas that may otherwise not be reached by a rigid device.

FIG. 9C illustrates, in the top view, how the flexible percutaneous surgical device 900 may be curved by turning the turning knob 916. For example, if the turning knob 916 is turned in the clockwise direction, as represented by an arrow 920, the deflection wire 904 attached to one end of the curvature guide 902 will move towards the tip 102 of the flexible percutaneous surgical device 900, as represented by an arrow 922. At the same time, the deflection wire 904 attached to the other end of the curvature guide 902 will move away from the tip 102 of the flexible percutaneous surgical device 900, as represented by an arrow 924. The movement of the deflection wire 904 forces the curvature guide 902 to turn, in this example, in the clockwise direction, as represented by an arrow 926. The curvature guide 902 in turn forces the protective sheath 106 to curve along with the new orientation of the curvature guide 902. The aforesaid set of movements enables the tip 102 to move, as represented by an arrow 928, to a new orientation. This new orientation of the tip 102, as well as the new curvature of the protective sheath 106, may enable the flexible percutaneous surgical device 900 to travel in a non-linear path.

FIG. 9D illustrates, in the side view, how the flexible percutaneous surgical device 900 may be curved by turning the turning knob 918. For example, if the turning knob 918 is turned in the clockwise direction, as represented by an arrow 930, the deflection wire 906 attached to one end of the curvature guide 902 will move towards the tip 102 of the flexible percutaneous surgical device 900, as represented by an arrow 932. At the same time, the deflection wire 906 attached to the other end of the curvature guide 902 will move away from the tip 102 of the flexible percutaneous surgical device 900, as represented by an arrow 934. The movement of the deflection wire 906 forces the curvature guide 902 to turn, in this example, in the clockwise direction, as represented by an arrow 936. The curvature guide 902 in turn forces the protective sheath 106 to curve along with the new orientation of the curvature guide 902. The aforesaid set of movements enables the tip 102 to move, as represented by an arrow 938, to a new orientation. This new orientation of the tip 102, as well as the new curvature of the protective sheath 106, may enable the flexible percutaneous surgical device 900 to travel in a non-linear path.

While the turning knobs 916 and 918 are turned in the clockwise direction in both FIGS. 9C and 9D, it is understood that the turning knobs 916 and 918 may also be turned in the counter-clockwise direction. As such, the motions depicted by the arrows 922, 924, 926, 928, 932, 934, 936 and 938 may be in completely different directions. It is further understood that the motions depicted in FIGS. 9C and 9D are two distinctive motions in planes that are orthogonal to each other; and that, by simultaneously turning both turning knobs 916 and 918, the protective sheath 106 of the flexible percutaneous surgical device 900 may curve in all directions in three dimensions, not merely on the two planes depicted in FIGS. 9C and 9D. Finally, it is understood that the turning knobs 916 and 918 may include a position locking mechanism such that the tensions, created on the deflection wires 904 and 906 by the turning motions of the turning knobs 916 and 918, may be locked during a specific surgical procedure.

It is understood that the percutaneous surgical device as described above can be used in conjunction with other surgery assistance devices such as a guide wire. For example, in order to maneuver better under the skin to get the percutaneous surgical device to a certain location, a small guide wire can be used to get to the desired location so that the relatively larger percutaneous surgical device can follow the guide wire to get to the same point. As such, the guide wire may be coupled to the percutaneous surgical device through a non-intrusive attaching mechanism such as an external tube that attaches to and runs along with the exterior of the sheath of the percutaneous surgical device. Similarly, the attaching mechanism can be on the interior wall of the sheath, and the guide wire can exit from the sheath/tip through the side opening or even another specifically designed opening.

The above disclosure provides many different embodiments or examples for implementing different features of the disclosure. Specific examples of components and processes are described to help clarify the disclosure. These are, of course, merely examples and are not intended to limit the disclosure from that described in the claims.

Although the invention is illustrated and described herein as embodied in various designs and methods for a percutaneous surgical device, it is nevertheless not intended to be limited to the details shown, since various modifications and structural changes may be made therein without departing from the spirit of the invention and within the scope and range of equivalents of the claims. Accordingly, it is appropriate that the appended claims be construed broadly and in a manner consistent with the scope of the disclosure, as set forth in the following claims.

1. A percutaneous surgical device comprising:
a tip with a side opening;
a protective sheath coupled to the tip, the sheath and tip housing at least one surgical instrument; and
a handle coupled to the sheath with at least one control means for controlling a moving element for controlling the surgical instrument to be exposed through the side opening of the tip.

2. The device of claim 1 wherein the surgical instrument is at a predetermined angle with reference to the sheath when exposed through the side opening as determined by the control means.

3. The device of claim 1 wherein the tip has a predetermined ramp for the surgical instrument to move along therewith.

4. The device of claim 1 wherein the tip has a securing means for securing the surgical instrument at a first point so that the surgical instrument rotates around the first point when controlled by a movement of the moving element.

5. The device of claim 4 wherein the moving element is secured to a second point on the surgical instrument for controlling the rotation of the surgical instrument.

6. The device of claim 1 further comprising a wedge coupled to the moving element positioned underneath the surgical instrument.

7. The device of claim 1 wherein the surgical instrument is secured to an inner wall of the sheath or the tip by a pole and the moving element is coupled to a predetermined point on the pole for controlling the exposure of the surgical instrument through the side opening.

8. The device of claim 1 wherein the surgical instrument travels along a predetermined angled moving path in the tip.

9. The device of claim 8 wherein the moving path is a rail.

10. The device of claim 9 wherein the rail has a rail cart for housing the surgical element thereon.

11. The device of claim 1 wherein at least a portion of the sheath is flexible for changing an orientation of the tip.

12. The device of claim 11 wherein the changing orientation of the tip is controlled by a curvature guide.

13. The device of claim 11 wherein the control means includes one or more turning knobs for controlling two deflection wires coupled to the curvature guide so that the turning of at least one turning knob changes the orientation of the tip.

14. The device of claim 1 wherein the control means includes a knob for pulling or pushing the moving element.

15. The device of claim 14 wherein the control means further includes a locking means for locking the knob.

16. The device of claim 14 wherein the control means further includes a distance marking means for measuring the movement of the moving element.

17. A percutaneous surgical device comprising:
a tip with a side opening;
a protective sheath coupled to the tip, the sheath and tip housing at least one surgical instrument; and
a handle coupled to the sheath with at least one control means for controlling a moving element for controlling the surgical instrument to be exposed through the side opening of the tip at a predetermined angle with reference to the sheath.

18. The device of claim 17 wherein the tip has a predetermined ramp for the surgical instrument to move along therewith.

19. The device of claim 17 wherein the surgical instrument is secured to an inner wall of the tip or sheath.

20. The device of claim 19 further comprising a wedge coupled to the moving element positioned underneath the surgical instrument.

21. The device of claim 19 wherein the surgical instrument is secured to the inner wall of the sheath or the tip by a pole and the moving element is coupled to a predetermined point on the pole for controlling the exposure of the surgical instrument through the side opening.

22. The device of claim 17 wherein the surgical instrument travels along a predetermined rail in the tip.

23. The device of claim 17 wherein at least a portion of the sheath is flexible for changing an orientation of the tip.

24. The device of claim 23 wherein the control means controls the turning of the flexible portion of the sheath for changing the orientation of the tip.

25. The device of claim 17 wherein the control means further includes a locking means for locking the moving element from moving.

26. The device of claim 17 wherein the control means further includes a distance marking means for measuring the movement of the moving element.

27. A percutaneous surgical device comprising:
a tip with a side opening;
a protective sheath coupled to the tip, the sheath and tip housing at least one surgical instrument; and
a handle coupled to the sheath with at least one control means for controlling a moving element for controlling the surgical instrument to be exposed through the side opening of the tip at a predetermined angle with reference to the side opening.

28. The device of claim 27 wherein the tip is replaceable with the surgical instrument.

29. The device of claim 27 wherein at least a portion of the sheath is flexible for changing an orientation of the tip while controlled by the control means.

30. The device of claim 27 wherein the sheath further includes an attached tube for securing a guide wire.
